(19) United States
(12) Patent Application Publication
(10) Pub. No.: US 2007/0089215 A1
(43) Pub. Date: Apr. 26, 2007

(54) BIFOCAL MAGNIFIER PLATE FOR USE IN WELDING

(75) Inventors: Barton A. Biche, Redwood City, CA (US); David C. Woodward, Redwood City, CA (US)

Correspondence Address: WARREN SKLAR;RENNER, OTTO, BOISSELLE & SKLAR, LLP, 1621 EUCLID AVE, 19TH FL, CLEVELAND, OH, 44115, US

(73) Assignee: LIGHTSWITCH SAFETY SYSTEMS, INC., Mountain View, CA (US)

(21) Appl. No.: 11/553,034

(22) Filed: Oct. 26, 2006

Related U.S. Application Data
(60) Provisional application No. 60/730,541, filed on Oct. 26, 2005.

(51) Int. Cl. A61F 9/06

(52) U.S. Cl. 2/8.2

(57) ABSTRACT

A protective lens assembly includes a protective lens, such as an auto-darkening lens, having a first viewing portion and a second viewing portion. One of the first or second viewing portions includes a magnifier plate, allowing the viewer to view magnified images through one of the first or second viewing portions and unmagnified images through the other of the first or second viewing portions. The lens assembly allows the viewer to perform close-up tasks and other tasks without having to reconfigure the lens assembly between tasks.

### TECHNICAL FIELD

The present application claims the benefit of U.S. Provisional Application Ser. No. 60/730,541, filed Oct. 26, 2005, the disclosure of which is herein incorporated by reference in its entirety.

### TECHNICAL FIELD

The present invention relates generally to protective lenses, such as lenses for welding masks, and, more particularly, to a protective lens that includes a bifocal magnifier plate for use in welding and the like.

### BACKGROUND

Welding helmets have been used in the past to protect the eyes and face of a person doing welding (hereinafter referred to as a welder) from the very bright light occurring during welding, e.g., emanating from the welding arc, and from possible other particles that may be flung toward the welder during welding. Early welding helmets included a lens through which a welder would view the workpiece being welded and a protective shield material, such as a metal, plastic or other solid material, that contained the lens and protected the welder's face from light emitted by the welding operation and from particles. Typically, the lens was a material that would transmit a relatively small amount of the incident light and, thus, when the welding was occurring, would permit enough light to pass to the welder's eyes to observe the welding operation while blocking a substantial amount of the light occurring during welding so that the eyes would not be injured.

Magnifier lenses have been used with welding helmets for many years to allow welders, especially older welders, to better focus on close-up work or more detailed work. Traditional welding magnifier lenses cover the entire viewing area of, for example, a standard 2 inch by 4.25 inch welding glass. While the use of a magnifier lens that covers the entire viewing area of the welding glass is preferred for close-up work, it is not suitable for most other activities.

In conventional designs, the limitations of having a magnifier lens cover the entire field of view are reduced with fixed dark welding glass, because the welding hood (and magnifier lens therein) only is in place during welding, which is a close-up activity, and does not interfere with normal vision tasks when the welding hood is flipped up away from the welder's eyes. However, with the growing popularity of auto-darkening welding lenses or filters, welders tend not to lift their helmets as often for non-welding tasks, but still require non-modified, e.g., non-magnified, vision for these tasks. In addition, in welding situations that require filtered air respirators to be worn, it might not be possible to lift the welding hood at all.

### SUMMARY

The present invention provides a protective lens assembly that includes a protective lens having a first viewing portion and a second viewing portion. One of the first or second viewing portions includes a magnifier plate, allowing the viewer to view magnified images through one of the first or second viewing portions and unmagnified images through the other of the first or second viewing portions. The lens assembly allows the viewer to perform close-up tasks and other tasks without having to reconfigure the lens assembly between tasks.

According to one aspect of the invention, a protective lens assembly includes a protective lens having a viewing area and a magnifier plate disposed adjacent a lower portion of the viewing area of the protective lens.

According to another aspect of the invention, an eye protection device includes a protective lens assembly having a first portion and a second portion, the protective lens assembly including a magnifier plate such that a viewer views unmagnified images through the first portion and magnified images through the second portion.

According to another aspect of the invention, a protective welding lens assembly includes a protective lens and a magnifier plate coupled to the protective lens, the magnifier plate having an upper portion and a lower portion, wherein the lower portion includes a magnifier lens such that a viewer views unmagnified images through the upper portion and magnified images though the lower portion.

According to another aspect of the invention, a welding helmet including a protective lens assembly including a bifocal magnifier plate is provided.

According to another aspect of the invention, a bifocal magnifier plate for use in a welding helmet is provided. The magnifier plate includes an upper portion and a lower portion, the lower portion including a magnifying lens such that a wearer of the welding helmet views unmagnified images through the upper portion and magnified images through the lower portion.

According to another aspect of the invention, a welding helmet includes an auto-darkening lens having a viewing area and a magnifier plate coupled to the auto-darkening lens such that a viewer views unmagnified images through an upper portion of the viewing area and magnified images though a lower portion of the viewing area.

According to another aspect of the invention, a protective apparatus for at least a portion of the face of a wearer includes a protective shield positionable with respect to the face of a wearer, a viewing area to permit viewing through the shield, and a magnifier plate adjacent a portion of the protective shield, the magnifier plate being positioned such that the wearer views unmagnified images through an upper portion of the viewing area and magnified images though a lower portion of the viewing area.

These and other objects, features, advantages and functions of the invention will become more apparent as the following description proceeds.

### BRIEF DESCRIPTION OF THE DRAWINGS

These and further features of the present invention will be apparent with reference to the following description and drawings, which are somewhat schematic and are not necessarily to scale, and wherein:

FIG. 1 is a schematic side view, broken away, of a bifocal welding lens assembly in a welding helmet in place on the head of a wearer;

FIG. 2 is a schematic side view of a bifocal lens assembly in accordance with the present invention;

FIG. 3 is a schematic side elevation view of a bifocal lens assembly in a respirator, space helmet or the like;

FIG. 4 is a schematic side elevation view of a bifocal lens assembly in a protective shield face mask; and

FIG. 5 is a schematic side elevation view of a bifocal lens assembly in goggles.

### DESCRIPTION

In the description that follows, like components have been given the same reference numerals, regardless of whether they are shown in different embodiments. To illustrate aspects of the present invention in a clear and concise manner, the drawings may not necessarily be to scale and certain features may be shown in somewhat schematic form. Features that are described and/or illustrated with respect to one embodiment may be used in the same way or in a similar way in one or more other embodiments and/or in combination with or instead of features of the other embodiments.

Aspects of the present invention are directed to a protective lens assembly that includes a bifocal magnifier plate. The lens assembly includes a protective lens, such as an auto-darkening lens, having a first viewing portion and a second viewing portion. One of the first or second viewing portions includes a magnifier plate, allowing the viewer to view magnified images through one of the first or second viewing portions and unmagnified images through the other of the first or second viewing portions. The lens assembly allows the viewer to perform close-up tasks and other tasks without having to reconfigure the lens assembly between tasks.

While exemplary embodiments of the invention will be described in connection with an automatic darkening lens (e.g., a lens that is able to operate automatically to control transmission of light through the lens), it will be appreciated that the invention is also applicable to embodiments in which the lens has a fixed shade for light transmission (e.g., a fixed dark welding glass).

In addition, while exemplary embodiments of the invention will be described in connection with a lens assembly used in a welding helmet for eye protection therein, it will be appreciated that the various features of the invention may be used in conjunction with other devices and functions. For example, the lens or lens assembly may be used in a welding helmet, and the lens may be used in other types of devices, such as goggles, spectacles, face masks, e.g., for industry (such as in an industrial plant or to protect outdoor or indoor electrical workers), for dentistry to protect the face of a dentist in the operative, respirator systems, nuclear flash eye protection devices, and other types of helmets and other eye-protection devices, etc. Such devices usually are employed to protect the face or the eyes of a person, as is known, for example, in the field of welding as well as other fields. Further, the lenses may be used in various other places to protect workers from bright light that could present a risk of injury.

In the description herein, reference will be made to a lens or lens assembly (also sometimes referred to as “welding lens,” “welding filter,” “shutter,” and the like), and to an automatically darkening lens (sometimes referred to as an auto-darkening lens or an auto-darkening filter) that is able to operate automatically to control the transmission of light through the lens. The lens may be a light shutter type of a device that is able to control light transmission without distorting, or at least with relatively minimal distortion of, the light and the image characteristics carried by the light or represented by the light. Therefore, when a person looks through the lens, the image seen would be substantially the same as the image seen without the lens, except that the intensity of the light transmitted through the lens may be altered depending on the operative state of the lens.

For the purposes of providing eye protection, usually a welding lens provides light blocking characteristics in the visible, infrared and ultraviolet wavelength ranges. The actual ranges may be determined by the components of the lens, the arrangement of those components, and so forth. One example of such a welding lens is described in U.S. Pat. No. 5,519,122. The lens assembly disclosed in that patent includes several liquid crystal cell light shutters, several plane polarizers, and a reflector or band pass filter, that are able to reflect ultraviolet and infrared electromagnetic energy and possibly also some electromagnetic energy in the visible wavelength range. The several liquid crystal cells, for example, may be birefringent liquid crystal cells sometimes referred to as surface mode liquid crystal cells or pi-cells.

As will be described further below, the present invention may be embodied in a variable optical transmission controlling device. Such a device is described in detail with respect to use in a welding helmet. However, it will be appreciated that such a device may be employed in other environments and in other devices and systems for controlling transmission of electromagnetic energy broadly, and, in particular, for controlling optical transmission. As used herein with respect to one example, optical transmission means transmission of light, i.e., electromagnetic energy that is in the visible spectrum and that also may include ultraviolet and infrared ranges. The features, concepts, and principles of the invention also may be used in connection with electromagnetic energy in other spectral ranges.

Examples of liquid crystal cells, lenses using them and drive circuits are described in U.S. Pat. Nos. 5,208,688, 5,252,817, 5,248,880, 5,347,383, and 5,074,647. In U.S. Pat. No. 5,074,647, several different types of variable polarizer liquid crystal devices are disclosed. Twisted nematic liquid crystal cells used in an automatic shutter for welding helmets are disclosed in U.S. Pat. Nos. 4,039,254 and Re. 29,684. Exemplary birefringent liquid crystal cells useful as light shutters in the present invention are disclosed in U.S. Pat. Nos. 4,385,806, 4,436,376, 4,540,243, 4,582,396, and Re. 32,521 and exemplary twisted nematic liquid crystal cells and displays are disclosed in U.S. Pat. Nos. 3,731,986 and 3,881,809.

Another type of liquid crystal light control device is known as a dyed liquid crystal cell. Such a dyed cell usually includes nematic liquid crystal material and a pleochroic dye that absorbs or transmits light according to orientation of the dye molecules. As the dye molecules tend to assume an alignment relative to the alignment of the liquid crystal structure or directors, a solution of liquid crystal material and dye placed between a pair of plates will absorb or transmit light depending on the alignment of the liquid crystal material. Thus, the absorptive characteristics of the liquid crystal device can be controlled as a function of applied electric field.

As is disclosed in several of the above patents, the respective shutters may have one or more operational characteristics (sometimes referred to as modes or states). One example of such an operational characteristic is the shade number; this is the darkness level or value of the shutter when it is in the light blocking mode. Another exemplary operational characteristic is the delay time during which the shutter remains in a dark state after a condition calling for the dark state, such as detection of the bright light occurring during welding, has ceased or detection thereof has terminated or been interrupted. Still another operational characteristic is sensitivity of one or both of the detection circuit or shutter to incident light, for example, to distinguish between ambient conditions and the bright light condition occurring during a welding operation and sensitivity also may refer to shutter response time or to the time required for the circuitry associated with the lens to detect a sharp increase in incident light (e.g., due to striking of the welding arc, etc.) and to switch the lens from the clear state to the dark state. Yet another characteristic, which may be considered an operational characteristic, is the condition of the battery or other power source for the shutter, such as the amount of power remaining, operational time remaining until the power source becomes ineffective, etc. In the past various operational characteristics of such shutters have been adjustable or fixed.

Dynamic operational range or dynamic optical range is the operational range of the lens between the dark state and the clear state, e.g., the difference between the shade numbers of the dark state and the clear state.

The disclosures of the patents identified herein are specifically incorporated in their entirety by reference.

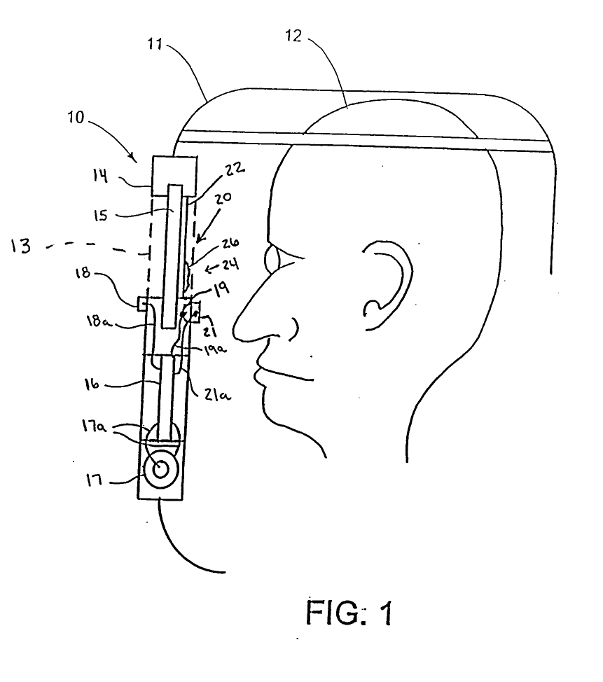

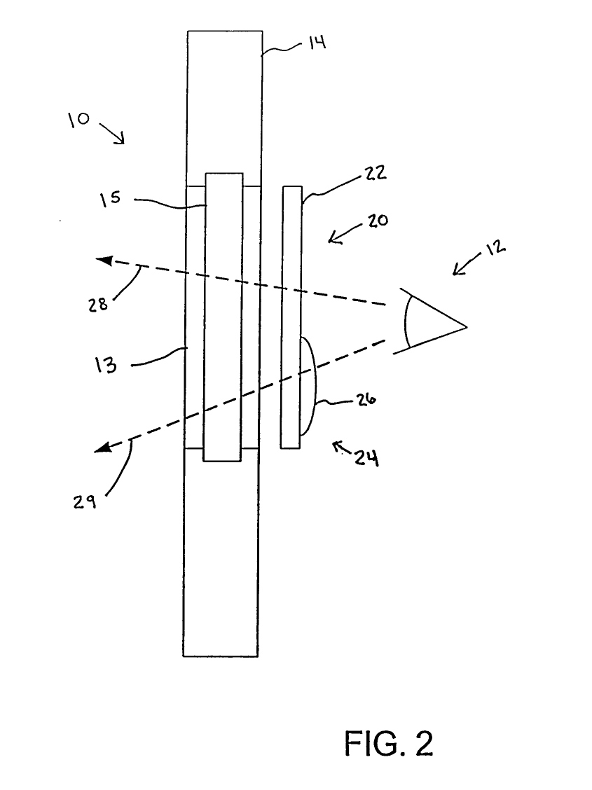

Referring now to the drawings, wherein like elements are referenced using like reference numerals, and initially to FIGS. 1 and 2, a lens assembly 10 (sometimes referred to as a bifocal or multi-focal lens assembly) is illustrated in a welding helmet 11 in position on the head of a wearer 12 (sometimes referred to as a user or, in one embodiment, a welder). In the illustrated embodiment, the lens assembly 10 is an auto-darkening lens assembly.

The lens assembly 10 includes a protective lens 13 that, in one embodiment, is made of a material that is strong enough to meet industrial standards of safety glasses for eye protection, such as industrial strength welding lens or shield glass. The lens assembly 10 further includes, for example, a support structure or housing 14, a variable light transmission shutter 15 mounted with respect to the support structure, operating circuitry 16 and a power supply 17.

The shutter 15 may be of the type disclosed in the above patents or some other suitable shutter that provides controllable variable light transmission capability and operation. Connections 17a couple the power supply 17 to provide power to the operating circuitry 16. Associated with the operating circuitry 16 is a photosensor 18, which is coupled to the operating circuitry by connections 18a, to sense occurrence of or a condition requiring a need for the auto-darkening lens assembly 10 to darken or to lighten, e.g., to decrease light transmission during welding or to increase light transmission in the absence of welding. The operating circuitry 16 operates the auto-darkening lens assembly to various conditions of light transmission. Several control buttons and switches schematically shown at 19 are coupled by connections 19a to the operating circuitry 16 and may be operated by the wearer 12 to turn on the operating circuitry 16 to operate the shutter 15, e.g., to adjust the desired degree of shade, to set a delay time, to set sensitivity, etc. As an example, the switches 19 may be membrane switches. One or more of the switches 19 may be a variable resistor or other adjustable electrical or electronic component; several non-limiting examples include a potentiometer, a stepped resistance, resistor or capacitor, respective capacitors, etc. The operating circuitry 16, power supply 17, photosensor 18, and buttons and switches 19 may be mounted on, in or part on and part in the support structure or may be otherwise located, as may be desired.

The lens assembly 10 includes a magnifier plate 20, such as a bifocal or a multi-focal magnifier plate, that is mounted on or within the protective lens 13. The magnifier plate can include a first or upper portion 22 and a second or lower portion 24 that includes a magnifier lens 26. In the embodiment illustrated schematically in FIG. 2, the magnifier plate 20 is configured such that a wearer or viewer 12 views unmagnified images through the upper portion 22 (along arrow 28) and magnified images through the lower portion 24 (along arrow 29 through the magnifier lens 26). In this embodiment (as well as others), use of a lens assembly including a magnifier plate provides one or more advantages in that the wearer would be able to perform close-up tasks (e.g., tasks in which magnified vision would be useful) as well as other tasks without having to remove the welding helmet and/or reconfigure the lens assembly between tasks.

The magnifier plate 20 can be configured in a number of ways without departing from the scope of the present invention. For example, the magnifier plate can be made of a separate plate mounted on or within the protective lens, such that the magnifier plate is mounted on the side of the protective lens that is closer to the wearer, and farther from the external welding activity. Alternatively, the magnifier plate can be an integral portion of the protective lens, for example, disposed within the protective glass adjacent the shutter 15. The magnifier plate 20 can be configured similar to a standard bifocal lens, a blended bifocal lens (also referred to as a continuous gradient bifocal lens), a tri-focal lens, and the like. In one embodiment, the first or upper portion of the magnifier plate is configured to assist a viewer with distance viewing (e.g., to assist with nearsightedness), while the second or lower portion of the magnifier plate includes a magnifying lens (e.g., to assist with farsightedness). Such a construction may be useful for a wearer who requires correction to help with both close-up and distance viewing. In another alternative embodiment, the first or upper portion of the magnifier plate includes a magnifier lens, while the second or lower portion of the magnifier plate does not. In yet another alternative embodiment, the magnifier plate includes only the lower portion (or the portion that includes the magnifier lens).

The magnifier plate 20 can be made of a material having a strength that is suitable for the position of the magnifier plate. For example, if the lens assembly is configured such that the magnifier plate is likely to be exposed to welding activity and particles associated therewith, the magnifier plate would be made of a material that meets industrial standards of safety glasses for eye protection.

Returning to FIG. 1, which includes an auto-darkening lens assembly 10 in the welding helmet 11, a wearer 12 may turn on the power and set the desired dark shade of the shutter 14 by using the buttons and switches 19. The wearer then puts the welding helmet 11 on the head with the shutter in front of the eyes for viewing work. The shutter 15 may be in its relatively clear or high light transmission condition (or state) to allow the wearer to view the work; and upon sensing occurrence of welding, the photosensor 18 indicates the same to the operating circuitry to cause the shutter to assume a dark or relatively reduced light transmission condition (or state). When welding ceases, the operating circuitry allows the shutter to return to the relatively clear condition.

Indicators 21 indicate operating conditions of the auto-darkening lens assembly 10. The indicators 21 may be coupled, as at 21a, to the operating circuitry or to some other device that operates the indicators. Examples of operating conditions may include, without limitation, the current shade or light transmitting condition of the shutter 15, e.g., is it clear or dark; reserve power supply power level, e.g, how much charge remains in the power supply (such as a battery) before becoming unable to supply adequate power to the operating circuitry 16 to operate the shutter 15; whether power from an external source is connected for operating the auto-darkening lens; whether the auto-darkening lens assembly 10 is on, e.g., is receiving power to the operating circuitry 16; what shade level has been set, e.g., by the buttons and switches 19; what delay time and/or sensitivity has been set, e.g., by the buttons and switches 19; etc.

The indicators 21 may be of the type that provide a light output. For example, each indicator may be a light emitting diode, an organic light emitting diode, an incandescent bulb, a combination of a light source and a light modulating device, such as a liquid crystal light modulator, or other type of device that provides a light output or indication based on light in response to an appropriate energization. The light output may be the generating or emitting of light by a given light source or it may be modulation of the light from a light source. The light output may be white, may be of a given color, or may be of different respective colors.

Operation of the indicators 21 may be provided by the operating circuitry 16. For example, the operating circuitry may provide respective signals and, if needed, power to cause respective indicators to provide a light output, to modulate light from a light source, to provide respective colors of light, etc. Such respective signals from the operating circuitry 16 may indicate the above-mentioned operating conditions of the auto-darkening lens assembly 10 or other information that may be of interest, useful or needed by the wearer 12.

Location of the indicators 21 is such that they would generally be in the peripheral field of view of the wearer 12 when the wearer is wearing the auto-darkening lens in a usual operative position with respect to the eyes of the wearer 12 and the wearer is looking through the shutter 15. The indicators 21 may be mounted on the support structure or housing 14 or may be mounted elsewhere, provided in use with the auto-darkening lens assembly 10 in place before the eyes of the wearer 12, the indicators generally are in the peripheral field of view of the wearer. With the auto-darkening lens assembly 10 before the eyes of the wearer, the indicators 21 would be so close to the face of the wearer that it would be difficult, if not impossible, for the wearer to focus an eye on the indicators as to see them clearly with good focus. As is illustrated in FIG. 1, the indicators 21 are at the bottom of the auto-darkening lens 10 and are mounted on the support structure 14.

Energization of the indicators 21 may be effected by inputs from the operating circuitry 16, as was mentioned above. For example, the operating circuitry may include a monitor to detect the amount of power (power reserve) remaining in a battery power supply for the auto-darkening lens; and in response to that detection the operating circuitry may illuminate a given indicator 21 or several indicators to indicate there is adequate power, the amount of power, e.g., the amount of time left before power runs out, that power will run out shortly, etc. If the power were to run out shortly, it would be desirable for the wearer to plan to stop working, e.g., welding, shortly and in any event before the power runs out, thereby to avoid the possibility that the work would be continued while the eyes would be unprotected by the dark state of the shutter 15. The operating circuitry 16 may provide an input to one or several indicators to indicate the pre-set shade level to which the shutter 15 will be operated when it is in the dark state, or to indicate response speed of the shutter, sensitivity level of the shutter, e.g., sensitivity of the photosensor 18 and operating circuitry to cause the shutter to be operated in the dark condition. The operating circuitry 16 may provide an input to one or several indicators to indicate that the shutter 15 is in a clear state condition or a dark state condition; this is valuable information because sometimes it is not possible for the wearer to recognize the condition of the shutter 15 by only looking through the shutter.

It will be appreciated that the operating circuitry 16 may have suitable detectors, operating software or firmware, and components, etc., to be able to determine which signals are to be directed to which indicators 21 to effect energization of such indicator(s) and the manner of energization, e.g., brightness, flashing, etc. The operating software or firmware may be written and the particular components of the operating circuitry 16 may be constructed by a person or persons having ordinary skill in the art based on the description herein. Moreover, as is described in the above-referenced and incorporated patents, various auto-darkening lenses and control systems are shown, and the present invention may be used, if desired, therewith.

As discussed above, the lens assembly 10, including a bifocal or multi-focal magnifier plate, can be used in connection with a number of eye protection devices, including, but not limited to a welding helmet, goggles, spectacles, face masks, e.g., for industry (such as in an industrial plant or to protect outdoor or indoor electrical workers), for dentistry to protect the face of a dentist in the operative, respirator systems, nuclear flash eye protection devices, and other types of helmets and other eye-protection devices.

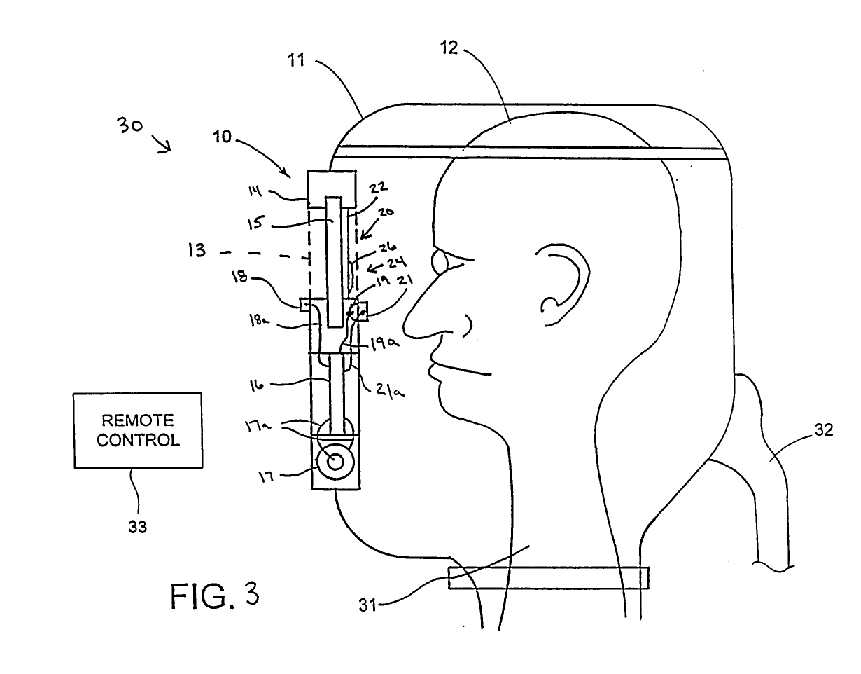

Referring to FIG. 3, a respirator 30 is illustrated schematically. The respirator 30 includes, by way of example, an auto-darkening lens assembly 10 mounted in a helmet structure 11 that fully encloses the head of a wearer 12, and may be sealed to the neck of the wearer at 31 or may be part of an overall enclosure, such as a diving suit, space suit, or other suit typically used for entering hazardous environments. One or more tubes 32 provide air flow and, if desired, provide for exhausting the products of breathing with respect to the interior of the helmet structure 11. The auto-darkening lens assembly 10 includes a magnifier plate 20 as is described above. In this embodiment (as well as others), use of a lens assembly including a magnifier plate provides one or more advantages in that the wearer would be able to perform close-up tasks (e.g., tasks in which magnified vision would be useful) as well as other tasks without having to remove the respirator (which may not be possible to do) and/or reconfigure the lens assembly between tasks.

The use and operation of the indicators 21 for the auto-darkening lens 10 in the respirator 30, etc., may be the same as is described above. If desired, a separate remote control schematically illustrated at 33 may be coupled by wire, radio signals, etc., to the operating circuitry 16 to allow the wearer of the respirator 30 and auto-darkening lens assembly 10 to adjust the operative conditions, settings, etc., of the auto-darkening lens by manual or other operation of the wearer. Since it would be difficult and sometimes inadvisable for the wearer briefly to remove the auto-darkening lens assembly 10 and respirator 30 to make such adjustments while wearing the respirator, the ability to make adjustments to settings of the auto-darkening lens assembly 10 remotely may be advantageous. Furthermore, being able to discern the settings being made by remote control, perhaps by another person, as the user discerns the indicators 21 in the user's peripheral field of view, allows the user to confirm that proper settings are being made.

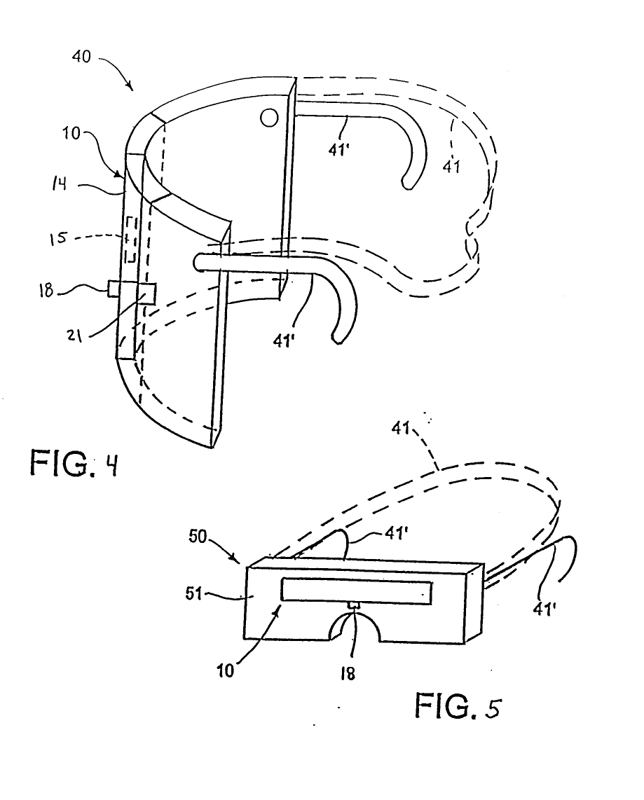

Briefly referring to FIG. 4, an auto-darkening lens assembly 10, including a magnifier plate as is described above, is illustrated in a dental mask or face protector 40. The dental mask 40 may be clear plastic or other material or it may have a light blocking characteristic. With the auto-darkening lens 10 assembly, including a magnifier plate, for example, mounted in the dental mask 40, e.g., to a support structure 14 thereof, and the dental mask 40 mounted on the head of a dentist, e.g., being held in place by a strap 41 or temple pieces 41′ so as place the mask and the auto-darkening lens assembly 10 in front of the face, the shutter 15 of the auto-darkening lens may be operated to protect the eyes of a dentist or dental technician from bright light and/or from light of a particular wavelength or wavelengths, e.g., ultraviolet used to cure a dental material, that might otherwise be harmful to the eyes, while the shutter still allows viewing therethrough absent such bright or possibly harmful light. The face protector 40 and the auto-darkening lens assembly 10 also protect the face from particulates, fluids, etc. during dental procedures. Similarly, the dental mask 40 with the auto-darkening lens assembly 10, for example, may be used by a surgeon conducting surgery, an industrial worker carrying out various tasks, and for other purposes, etc., to provide similar utility would be provided for a dentist. Use of a lens assembly 10, including a magnifier plate as is described above, allows the wearer to perform close-up tasks (e.g., tasks in which magnified vision would be useful) as well as other tasks without having to remove the mask (which may not be possible to do) and/or reconfigure the lens assembly between tasks.

Briefly referring to FIG. 5, an auto-darkening lens assembly 10, including a magnifier plate as is described above, is shown in goggles 50. The goggles may be used for many different purposes, such as for protection in industrial environments, for racing automobiles or airplanes in an open cockpit, for observing experiments or other conditions in which bright light, even nuclear flash, may occur, etc. The goggles include a support structure 51, sometimes referred to as a frame, with appropriate components, as is schematically illustrated in FIG. 5, to allow the goggles to be worn on the head of a person. The auto-darkening lens assembly may be mounted in such a support structure for use generally as was described above.

The goggles 50 may be in the form of eyeglasses that can be worn by a wearer. Such eyeglasses would include a support structure, such as a lens holder of an eyeglass frame, temple pieces to hold to the ears of a wearer, etc., as is schematically illustrated in FIG. 5. The eyeglasses 50 may use a lens 10 (or 10′ or 10″) that is a safety lens, which has a characteristic capable of meeting industrial standards of safety glasses for eye protection in an industrial plant. Use of a lens assembly 10, including a magnifier plate as is described above, allows the wearer to perform close-up tasks (e.g., tasks in which magnified vision would be useful) as well as other tasks without having to remove the goggle or safety glasses (which may not be possible to do) and/or reconfigure the lens assembly between tasks.

It will be appreciated that although the invention is described with respect to one or more embodiments, the scope of the invention is limited only by the claims and equivalents thereof. It also will be appreciated that if the invention is described with respect to several embodiments, features of a given embodiment also may be used with one or more other embodiments.

To the accomplishment of the foregoing and related ends, the invention, then, includes the features hereinafter described in the specification and particularly pointed out in the claims, the following description and the annexed drawings setting forth in detail certain illustrative embodiments of the invention, these being indicative, however, of but several of the various ways in which the principles of the invention may be suitably employed.

### INDUSTRIAL APPLICATION

It will be appreciated that the aforementioned embodiments of the present invention may be used to provide a lens assembly for use in various eye protection devices that allows a wearer to perform close-up tasks and other tasks without having to reconfigure the lens assembly between tasks.

What is claimed is:

1. A protective lens assembly comprising:
a protective lens having a viewing area; and
a magnifier plate disposed adjacent a lower portion of the viewing area of the protective lens.

2. The protective lens assembly of claim 1, wherein the protective lens is a variable transmission lens.

3. The protective lens assembly of claim 2, wherein the protective lens is an auto-darkening lens.

4. The protective lens assembly of claim 1, wherein the magnifier plate includes a magnifying lens.

5. The protective lens assembly of claim 1 in combination with a helmet.

6. The protective lens assembly of claim 5, wherein the helmet is a welding helmet.

7. The protective lens assembly of claim 5, wherein the helmet is part of a respirator.

8. The protective lens assembly of claim 5, wherein the helmet is a space helmet.

9. The protective lens assembly of claim 1 in combination with goggles.

10. An eye protection device comprising:
a protective lens assembly having a first portion and a second portion, the protective lens assembly including a magnifier plate such that a viewer views unmagnified images through the first portion and magnified images through the second portion.

11. The eye protection device of claim 10, wherein the first portion is an upper portion and the second portion is a lower portion.

12. The eye protection device of claim 10, wherein the magnifier plate is a bifocal magnifier plate having a magnifying lens.

13. The eye protection device of claim 10, wherein the magnifier plate is a multi-focal lens including a magnifying lens portion adjacent the second portion.

14. The eye protection device of claim 10, wherein the first portion is configured to assist with distance viewing.

15. The eye protection device of claim 14, wherein the first portion is configured to assist with nearsightedness and the second portion is configured to assist with farsightedness.

16. The eye protection device of claim 10, wherein the protective lens is a variable transmission lens.

17. The eye protection device of claim 10, wherein the protective lens is an auto-darkening lens.

18. A welding helmet including the eye protection device of claim 10

19. A filtered air respirator including the eye protection device of claim 10.

20. Goggles including the eye protection device of claim 10.

21. A protective welding lens assembly comprising:
a protective lens; and
a magnifier plate coupled to the protective lens, the magnifier plate having an upper portion and a lower portion, wherein the lower portion includes a magnifier lens such that a viewer views unmagnified images through the upper portion and magnified images though the lower portion.

22. The protective welding lens assembly of claim 21, wherein the protective lens is a variable transmission lens.

23. The protective welding lens assembly of claim 21, wherein the protective lens is an auto-darkening lens.

24. A welding helmet including the protective lens assembly of claim 23.

25. A welding helmet including a protective lens assembly including a bifocal magnifier plate.

26. The welding helmet of claim 25, wherein the protective lens assembly includes a variable transmission lens.

27. The welding helmet of claim 25, wherein the the protective lens assembly includes an auto-darkening lens.

28. A bifocal magnifier plate for use in a welding helmet, the magnifier plate including an upper portion and a lower portion, the lower portion including a magnifying lens such that a wearer of the welding helmet views unmagnified images through the upper portion and magnified images through the lower portion.

29. A welding helmet comprising an auto-darkening lens having a viewing area and a magnifier plate coupled to the auto-darkening lens such that a viewer views unmagnified images through an upper portion of the viewing area and magnified images though a lower portion of the viewing area.

30. A filtered air respirator including the welding helmet of claim 29.

31. A protective apparatus for at least a portion of the face of a wearer, comprising:
a protective shield positionable with respect to the face of a wearer, a viewing area to permit viewing through the shield, and a magnifier plate adjacent a portion of the protective shield, the magnifier plate being positioned such that the wearer views unmagnified images through an upper portion of the viewing area and magnified images though a lower portion of the viewing area.

32. The apparatus of claim 31, wherein the protective shield includes an auto-darkening welding lens.

33. The apparatus of claim 31, wherein the shield is a helmet.

34. The apparatus of claim 33, wherein the helmet is a welding helmet.

35. The apparatus of claim 33, wherein the helmet is part of a respirator.
